(19) United States
(12) Patent Application Publication
(10) Pub. No.: US 2007/0282365 A1
(43) Pub. Date: Dec. 6, 2007

(54) Dilating Trocar

(76) Inventor: Sergey Popov, Beer Sheva (IL)

Correspondence Address: DANIEL J SWIRSKY, 55 REUVEN ST., BEIT SHEMESH, 99544, IL

(21) Appl. No.: 10/579,538

(22) PCT Filed: Nov. 11, 2004
(86) PCT No.: PCT/IL04/01035

§ 371 Date: Jan. 17, 2007

(30) Foreign Application Priority Data
Nov. 16, 2003 (IL) 158889

(51) Int. Cl. A61M 29/02

(52) U.S. Cl. 606/192

(57) ABSTRACT

Dilating trocar (40) (see FIG. 14) comprises cannula (42) containing carrier (51) with pivotable dilating members (49, 50) of small transversal dimensions protruding distally of the cannula. Obturator (63) is housed inside the carrier and has sharp element (70) with underspringed protective shield (71) protrudind distally of the dilating members. After inserting the closed dilating members into a small opening primary made in patient's body cavity wall (60) by sharp element (70), user removes the obturator, introduces an actuating rod into carrier (51) and forces apart the dilating members rotating them about axes (52, 53) and thereby stretching the primary opening to provide the cannula insertion therein. Finally, user removes the actuating rod, carrier and dilating members from cannula (42) remaining the cannula in patient's body.

### BACKGROUND

1. Field of the Invention

The present invention relates to surgical instruments designed for providing the access to patient's body cavity in the minimal invasive surgery (MIS). Specifically, it relates to the trocar devices designed for forming an opening in the body cavity wall and installing an access port therein for the performance of surgery operations.

2. Prior Art of the Invention

The general feature of the many modern trocars is the use of considerable operators axial force needed for the trocar advance into the patients body tissue. This leads to the operation inconvenience and creates the probability of the injury of patients internal organs by sharp trocar tip at the early moment of the trocar introduction into a patient's cavity. The increased tissue incision allows partial solving this problem. However, this solution is undesirable because of the increased injury of the patients tissue. The attempt of the solution of the indicated problems is disclosed in the article “Randomized Trial Comparing a Radially Expandable Needle System with Cutting Trocars” (2000 by JSLS, Journal of the Society of Laparoendoscopic Surgeons. Published by the Society of Laparoendoscopic Surgeons, Inc.; JSLS(2000)4:11-15). According to the article, a radially expandable needle system consists of an access-insufflation needle, a radially expandable polymeric sleeve, and a tapered blunt dilator/cannula. The surgeon inserts the needle into the abdomen and insufflates. The needle is then removed and reinserted with the sleeve. Thereafter, the surgeon removes the needle, leaving the sleeve in place, and a blunt dilator and cannula are inserted through the sleeve, thereby stretching the tissue of abdominal wall and enlarging the sleeve diameter. After removing the dilator, the cannula can be used for inserting laparoscopic instruments into the abdominal cavity. The disadvantage of this method is considerable spending the operation time caused with many operations needed for the cannula emplacement. Another disadvantage is the large axial force needed for inserting the dilator and cannula into initially small-sized sleeve. This force inevitably leads to great pressing onto patient's internal organs, which can be transformed into strokes due to uneven advance of the cannula within the sleeve. As a result, the patients internal organs can be contused by a distal edge of the cannula. Moreover, the necessary great axial force causes the inconvenience in a surgeon operation. Another disadvantage is the increased injury of the abdominal wall due to excessive stretch of the body tissue, leading to a tissue ruptures and forming an avulsive wound.

Another attempt is disclosed in U.S. Pat. No. 6,309,349 “Surgical retractor and stabilizing device” for the performance of closed-chest exploratory or surgical procedures on a patient. The surgical retractor is provided with two opposable blades to be inserted into previously made a surgical incision and forced apart by two control levers to form an access opening through which an instrument may be inserted into the thoracic cavity. In this invention, the axial force needed for the retractor emplacement is reduced to a minimum. The disadvantage of the invention is the increased injury of patient's body by the relatively large previous incision needed for inserting the blade edges and for forming the ovoid access opening. The latter is needed for preventing the body's tissue from substantial protrusion into the surgical instrument operation zone of the access opening on the open sides of the access opening. Carrying out the large through previous incision also is inconvenient and takes curtain time. Another disadvantage is the restricted application field of the invention, suitable for the thoracic surgery and non-applicable in the abdominal surgery.

### SUMMARY OF THE INVENTION

The objective of the present invention is substantial decrease of surgeon's axial force needed for the trocar emplacement.
Another objective is substantial decrease the patient's tissue injury.
Another objective is improving the operation convenience.
Another objective is enhancing the safety of the trocar operation.
Another objective is reducing the time needed for the trocar emplacement.
Another objective is providing the reliable and relatively inexpensive design of the trocar.

The above noted objectives are accomplished with a dilating trocar for forming a passageway in a body cavity wall between a body cavity and surroundings by stretching a through primary opening in the body cavity wall to the size of a complete opening. The dilating trocar comprises: a cannula having a tubular passageway portion and a housing disposed at the proximal end of the passageway tubular portion; a dilating means designed for stretching the primary opening and having a distal portion; which at least from the beginning to the completion of stretching the primary opening is located inside the body cavity, and a dilating portion of changeable geometry disposed adjacent to the distal portion proximally of it, distally of the passageway portion and having dilating surfaces. The dilating means has a first position and a second position. In the first position the distal portion is located inside the body cavity, the dilating portion is placed into the primary opening so that at least by the beginning of stretching the primary opening the walls of the primary opening along its full length are disposed within the axial limits of the dilating portion, the dilating surfaces are faced the walls of the primary opening and the transversal dimensions of the dilating portion and distal portion measured at least in the same plane parallel to the longitudinal axis of the dilating trocar are considerably less than the outer diameter of the passageway portion to provide inserting the dilating portion into the primary opening with an insignificant resistance offered by the body tissues and without substantial stretching the primary opening. In the second position the transversal dimensions of the dilating portion are effectively bigger than its correspondent dimensions in the first position, and the transition from the first position to the second position leads to stretching the primary opening to the size approximately equal to the size of the complete opening and sufficient for introducing therein the passageway portion. The dilating means also includes an actuating means for the transpositions of the dilating means from the first position to the second position. The dilating portion includes at least two dilating members connected to a dilating member carrier at its distal end and movable relative to each other at least in the transversal direction with respect to the longitudinal trocar axis, and at least one of the dilating members has a movable connection to the dilating member carrier. The actuating means has the movable thread connection with the cannula allowing forcing apart the dilating members without creating the axial force displacing the body cavity wall with respect to patient's internal organs. The dilating portion comprises an axial passage, wherein a removable obturator is housed during inserting the dilation portion into the primary opening. The obturator has an obturator handle disposed at the obturator proximal end and a penetrating means located at the obturator distal end and partly protruding distally of the distal portion of dilating means. The penetrating means comprises a sharp member, which serves for forming the primary opening and is provided with a protecting means including a movable protecting shield and a biasing spring. In version embodiment, the dilating portion includes a balloon expandable in the transversal direction with respect to the longitudinal trocar axis and an actuating means designed for expanding the balloon from the first position to the second position by its filling with fluid after its introducing into the primary opening.

First, he surgeon carries out the primary opening in the body cavity wall by the penetrating means and introduces the dilating portion into the primary opening. In doing so, the surgeon's axial force is substantially decreased due to the presence of the sharp element and the small transversal dimensions of the dilating portion. This improves the operation convenience. Moreover, piercing the body cavity wall and introducing the dilation portion, carried out as a single operation, substantially reduced the operation time and additionally improves the operation time. Therewith, the presence of the protective means, protecting the sharp element immediately after its entering the body cavity, and the insignificant penetrating axial force substantially enhance the safety of the trocar operation. Thereafter, the surgeon forces apart the dilating member by the actuating means stretching the primary opening without creating the axial force displacing the body cavity wall to the internal organs. The latter additionally enhances the operation safety eliminating the injury or contusion of the internal organs. Moreover, obtaining the needed opening by small incision of the tissue with its subsequent moderate stretching reduced to a minimum the tissue injury and creates the favorable conditions for the subsequent patients recovery. The dilating trocar design includes all the needed elements allowing carrying out the cannula emplacement as the complex of successive operations without an intermission and using additional instruments. These operations are completed with inserting the cannula passageway tubular portion into the complete opening and removing the dilating means from the cannula interior.

### BRIEF DESCRIPTION OF THE DRAWINGS

FIGS. 1 to 17 show the dilating trocar comprising a carrier removably housed inside the cannula and having two dilating members pivotably connected to the carrier by hinges, actuating means and sectional obturator subassembly independent of each other and sequentially installed into the carrier, wherein:

FIG. 1 shows the cannula.

FIG. 2 shows the cannula put together with the carrier having the dilating members in the first position.

FIGS. 3 to 5 show the carrier with two dilating members.

FIGS. 6, 7 show the sectional obturator subassembly.

FIG. 8 shows the lower section of the obturator subassembly comprising of a penetrating means.

FIG. 9 shows the upper section of the obturator subassembly.

FIG. 10 shows the dilating trocar comprising the cannula, sectional obturator and carrier with the dilating members in the first position.

FIG. 11 shows the actuating means.

FIG. 12 shows the dilating trocar comprising the cannula, actuating means and carrier with the dilating members in the first position.

FIG. 13 shows the dilating trocar comprising the cannula, actuating means and carrier with the dilating members in the second position.

FIGS. 14 to 17 show the process of dilating trocar emplacement.

FIG. 18 shows the carrier with the dilating members pivotably connected to the carrier by flexible links.

FIG. 19 shows the dilating trocar comprising a carrier removably connected to the cannula by the latch mechanism, having dilating members pivotably connected to the carrier by flexible links, and removable obturator and actuating means which are independent of each other and sequentially installed into the carrier.

FIG. 20 shows the dilating trocar comprising an intermediate sleeve removably connected to the cannula by the latch mechanism, having dilating members pivotably connected to the carrier by flexible links, and the carrier along with the actuating means and the obturator are simultaneously installed into the cannula through the intermediate sleeve.

FIGS. 21 to 27 show the dilating trocar comprising the carrier with one dilating member movable parallel to another immovable dilating member, the penetrating means disposed on the carrier, and the actuating means removably housed inside the carrier, wherein:

FIGS. 21 to 23 show the subassembly including the carrier with the dilating members and penetrating means, as well as the actuating means.

FIG. 24 shows the dilating trocar in the first position.

FIGS. 25, 27 show the dilating trocar in the second position.

FIG. 26 shows the cannula.

FIGS. 28 to 30 show the dilating trocar comprising the carrier removably housed inside the cannula and including the dilating portion in the form of an expandable balloon and the actuating means in the form of a syringe, wherein:

FIG. 28 shows the subassembly including the carrier and actuating means.

FIG. 29 shows the dilating trocar with the dilating portion in the first position.

FIG. 30 shows the dilating trocar with the dilating portion in the second position.

### DETAILED DESCRIPTION OF THE INVENTION

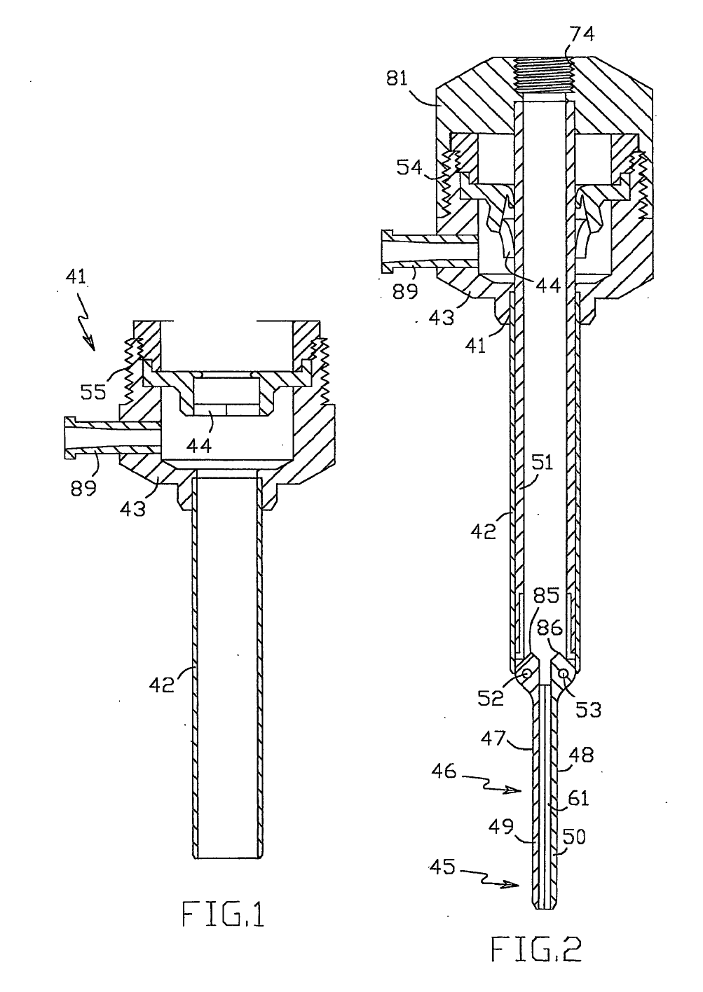

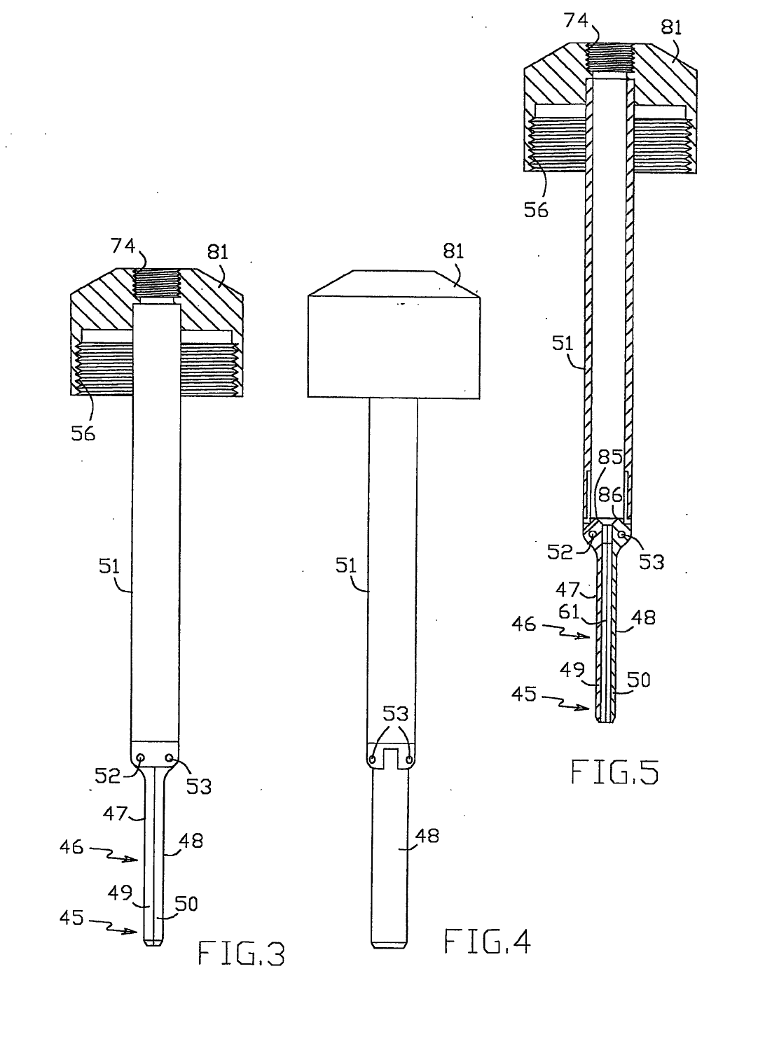

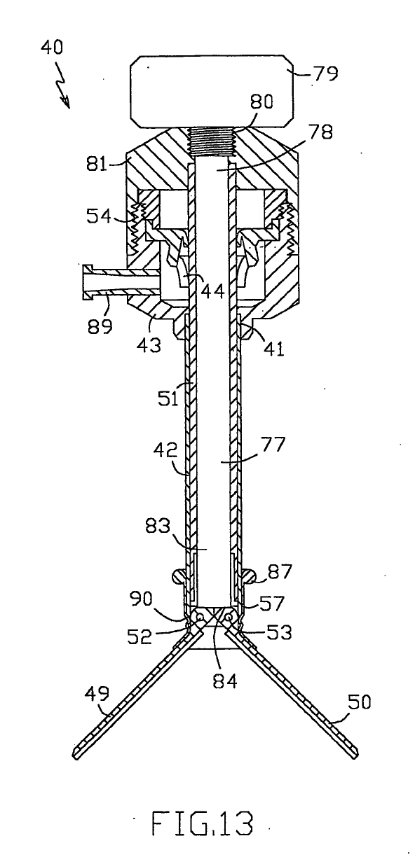

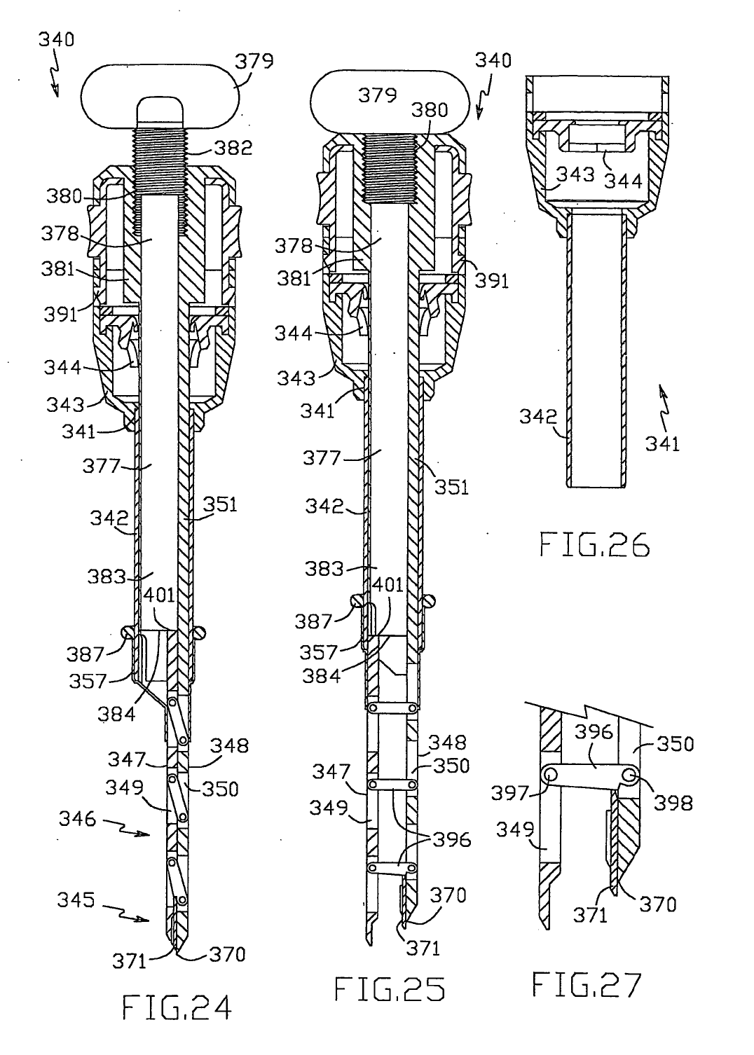

The detailed description of the present invention is offered with references made to the enclosed drawings in FIGS. 1 to 30.

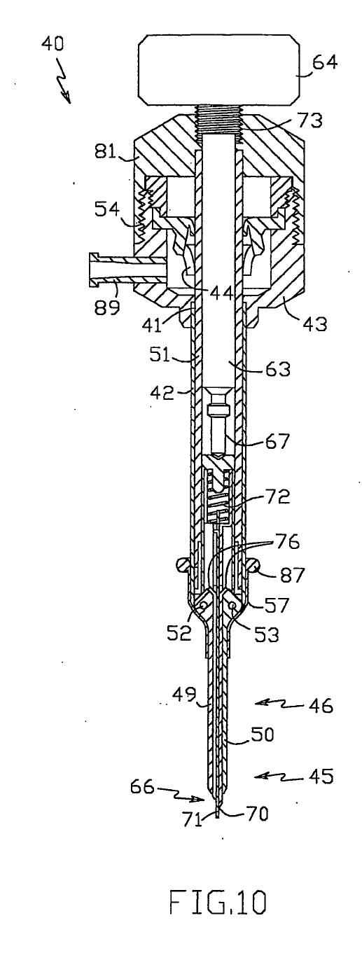

The dilating trocar 40, shown in FIGS. 1 to 17, comprises cannula 41 having tubular passageway portion 42 and housing 43 with sealing valve 44 disposed at the proximal end of passageway portion 42 and a dilating means including distal portion 45 and dilating portion 46 of changeable geometry having dilating surfaces 47, 48 and disposed adjacent to distal portion 45 proximally of it. Distal and dilating portions 45, 46 are made in the form of two dilating members 49 and 50 pivotably connected to carrier 51 by hinges 52 and 53. Carrier 51 (FIGS. 3 to 5) is designed for removable insertion inside passageway portion 42 and releasable engagement with cannula housing 43 by thread connection 54 (FIG. 2) including thread 55 on cannula housing 43 and thread 56 on carrier 51. There is sealing element 57 (FIG. 10) in the form of a resilient sleeve with flange 87 embracing the distal end of passageway portion 42 and proximal ends of dilating members 49, 50 and preventing the gas leakage from the body cavity at the early period of the dilating trocar emplacement.

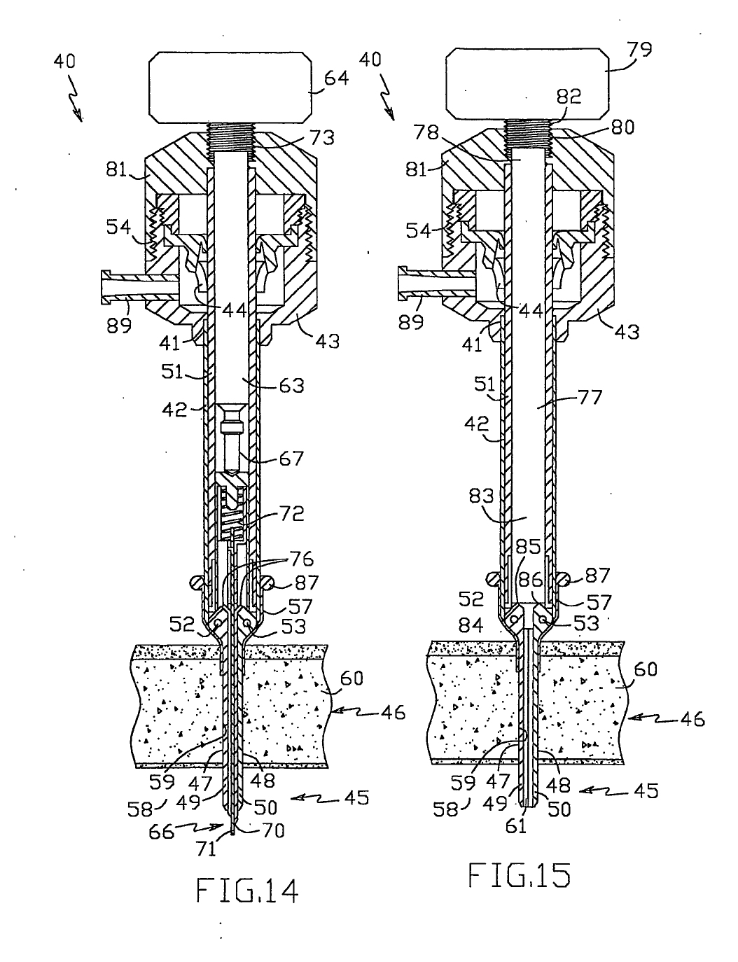

In the first position (FIG. 14), distal portion 45 is located inside body cavity 58, dilating portion 46 is placed into primary opening 59 in body cavity wall 60 so that at least by the beginning of stretching the primary opening 59 the walls of primary opening 59 along its full length are disposed within the axial limits of dilating portion 46, and dilating surfaces 47, 48 are faced the walls of primary opening 59. Therewith, the transversal dimensions of dilating portion 46 and distal portion 45 measured at least in the same plane parallel to the longitudinal axis of dilating trocar 40 are considerably less than the outer diameter of passageway portion 42 to provide inserting dilating portion 46 into primary opening 59 with an insignificant resistance offered by the body tissues and without substantial stretching primary opening 59. In the first position, dilating surfaces 47, 48 and a longitudinal trocar axis are substantially parallel to each other with the exception of certain small deviations from said parallelism not exceeding 5 degrees and needed for the auxiliary purposes.

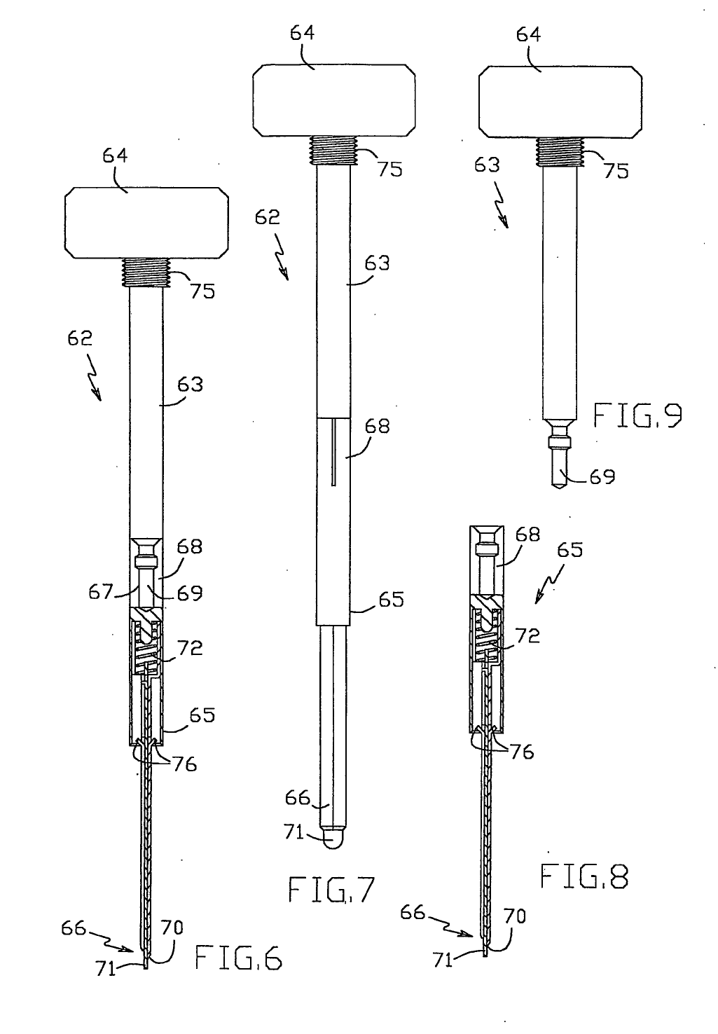

Dilating portion 46 and distal portion 45 have axial passage 61 (FIGS. 2, 5) wherein a guide is housed, which serves for inserting dilation portion 46 into primary opening 59. In dilating trocar 40, the guide is made as removable sectional obturator 62 shown in FIGS. 6 to 9. Obturator 62 consists of proximal section 63 including obturator handle 64 and distal section 65 including penetrating means 66 for carrying out primary opening 59. Section 63 and 65 are connected by collet connector 67 including collet bushing 68 and collet rod 69 eliminating the section disconnection when obturator 62 is located inside carrier 51 (FIG. 10) and allowing their easy disconnection when obturator is beyond carrier 51 (FIGS. 6, 7). Penetrating means 66 is located at a distal end of obturator 62 and, in the first position, protrudes distally of distal portion 45 (FIGS. 10, 14). Penetrating means has sharp element 70 for piercing the body cavity wall and the protective means designed for protecting the sharp element 70 and including shield 71 and biasing spring 72. The presence of sharp element 70 and the above indicated small transversal dimensions of distal and dilating portions 45, 46 provide the considerable decrease of the user's axial force, needed for performing primary opening 59 in body cavity wall 60 and inserting portions 45, 46 into primary opening 59. As a result, the operation convenience is improved. Immediately after entering body cavity 58, sharp element 70 is protected with shield 71, thereby preventing the internal organs from injury and enhancing the operation safety. The mentioned axial force decrease also decreases the probability of patient's injury. Obturator 62 and carrier 51 are releasably connected by thread connector 73 including thread 74 on carrier 51 and thread 75 on obturator 62. Therewith, collet connector 67 allows rotating obturator handle 64 to connect or disconnect obturator 62 and carrier 51, while obturator distal section 65 is non-rotatable. The use of obturator 62 with penetrating means 66 as a guide for inserting distal and dilating portions 45, 46 into primary opening 59, allows to perform forming primary opening 59 and inserting dilating portion 46 therein as a single operation, thereby reducing the operation time and additionally improving the operation convenience as compared with prior art (see the article “Randomized Trial Comparing a Radially Expandable Needle System with Cutting Trocars”). Obturator 62 also includes projections 76 designed for locking dilating members 49, 50 in the first position (FIGS. 10, 14).

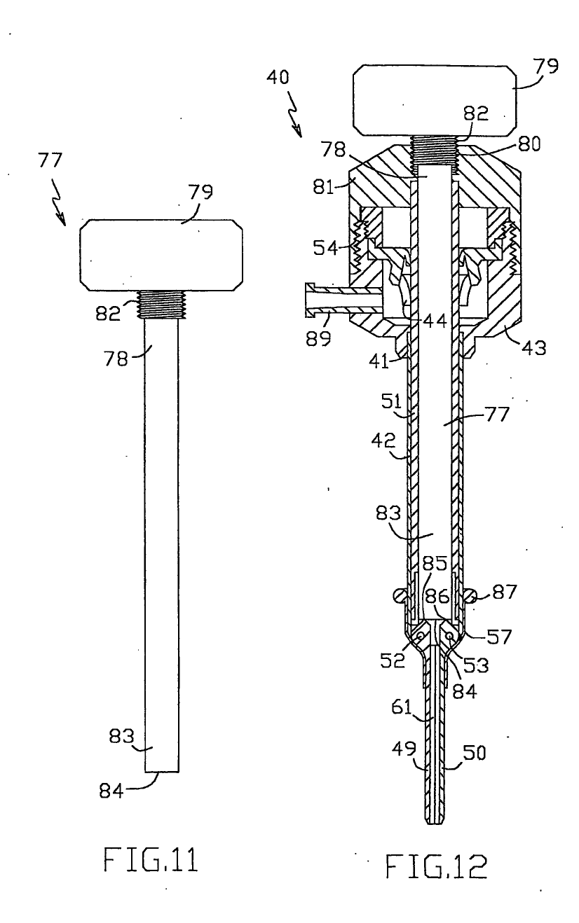

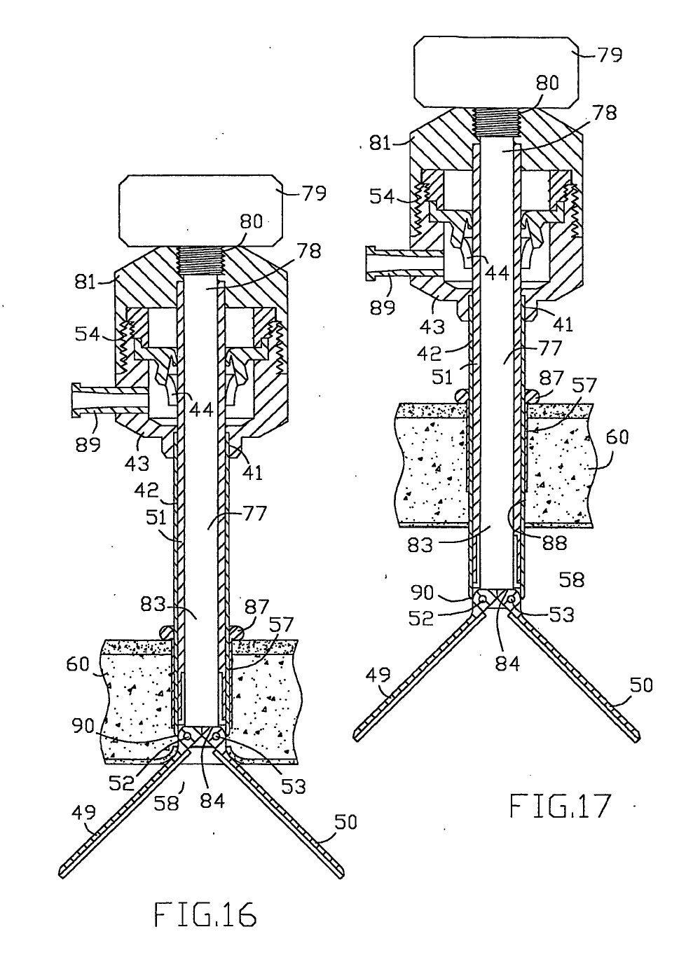

After inserting dilating portion 46 into primary opening 59, the surgeon removes obturator 62 along with penetrating means 66 from carrier 51 and installs actuating means 77 into carrier 51 (FIGS. 11, 12, 15). Actuating means 77 includes actuating member 78, actuating handle 79, and movable tread connection 80 with carrier housing 81 including thread 82 of actuating member 78. These elements 78, 79 and 80 form the actuator mechanism designed for the user's control of the transpositions of dilating members 49, 50. There is also the transmitting means for transmitting the control efforts from the actuator mechanism to dilating members 49, 50 including transmitting rod 83, which in dilating trocar 40 is made as a continuation of actuating member 78. Distal face 84 of transmitting rod 83 as well as proximal ends 85, 86 of dilating members 49, 50 are adapted for interacting with each other to force apart dilating members 49, 50 and release them by axial transposition of actuating member 78 and transmitting rod 83 relative to carrier 51. After inserting dilating portion 46 into primary opening 59 and before the commencement of stretching the primary opening 59, body cavity 58 is inflated by gas supplied through gas supplying means 89. Then, displacing of transmitting rod 83 in the distal direction is carried out to force apart dilating members 49, 50 and transpose them to the second position (FIG. 13), stretch primary opening 59, and partly introduce cannula passageway portion 42 (FIG. 16) into the stretched primary opening. Complete cannula emplacement is implemented by additional surgeon's axial force (FIG. 17). The obtained stretched primary opening is approximately equal to the size of the complete opening fully formed as a result of inserting cannula passageway portion 42 (FIG. 17). Therewith, the stretched primary opening may be somewhat less than complete opening 88 allowing introducing passageway portion 42 with some stress to obtain the tight joint of body wall 60 and passageway portion 42. Carrying out the complete opening by previous small tissue incision and subsequent its moderate stretching to needed size reduces to a minimum the patient's body injury. During the cannula insertion into the stretched primary opening, flange 87 of sealing element 57 is abutted against the skin of body cavity wall 60 preventing sealing element from inserting into body cavity 58.

After the cannula emplacement, actuating means 77 and thereafter carrier 51 along with dilating members 49, 50 are removed from cannula 41. In doing so, dilating members 49, 50 are reverted from the second position to the first position by the reverting means whose role fulfils distal edge 90 of cannula passageway portion 42. Edge 90 forces dilating members 49, 50 to revert to the first position during removing carrier 51 from cannula 41 after previous removing actuating means 77 from the zone of contact with dilating members 49, 50.

In version embodiment (not shown), the dilating members are pivotably connected to a distal end of the cannula passageway portion, and the actuating means is installed immediately into the cannula.

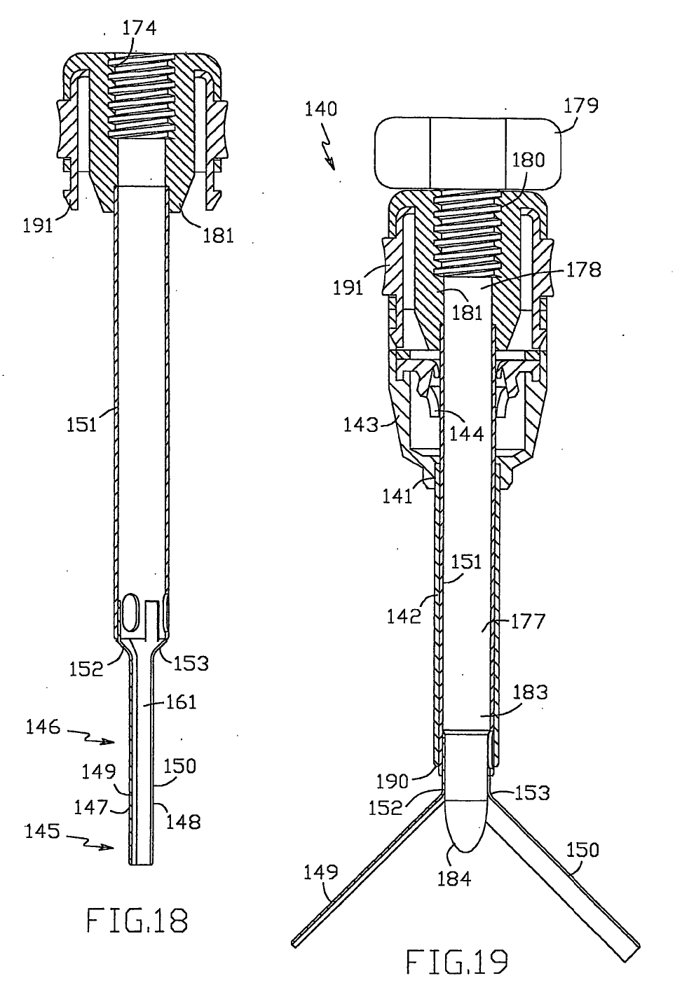

In version embodiment (FIGS. 18, 19), dilating trocar 140 includes dilating members 149, 150 pivotably attached to carrier 151 by flexible links 152, 153, and carrier housing 181 is connected with cannula housing 143 by controllable latch mechanism 191. The latter allows introducing carrier 151 into cannula 141 and withdrawing therefrom by surgeon's axial movement. The configuration of distal face 184 of transmitting rod 183 is adapted for interacting with flexible links 152, 153 to force apart dilating members 149, 150. Otherwise, the design and operation of dilating trocar 140 are identical to one of dilating trocar 40, and the designations of their identical parts have the same two last numerals.

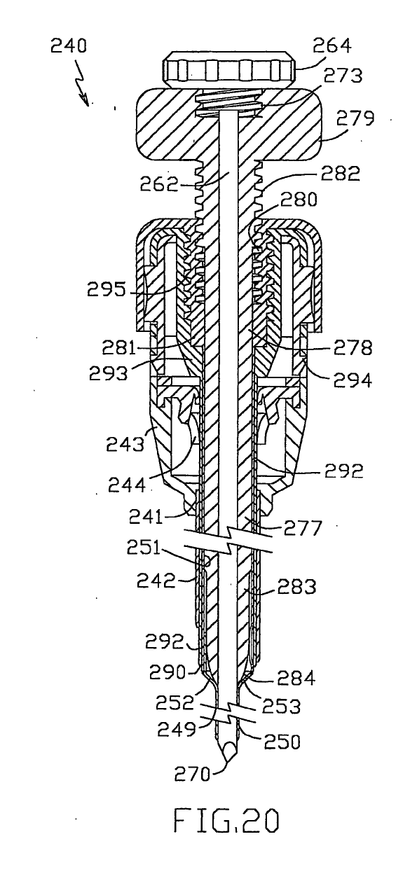

In another version embodiment, shown in FIG. 20, obturator 262 is removably housed inside actuating means 277 and connected with it by thread connection 273, actuating means 277 is removably housed inside carrier 251 and connected with carrier housing 281 by thread connection 280, and carrier 251, in turn, is removably housed inside intermediate sleeve 292 and connected with its housing 293 by thread connection 295. Intermediate sleeve 292 is removably housed inside cannula 241 and connected with cannula housing 243 by latch 294. Intermediate sleeve 292 allows adapting the unified set of the carrier, actuating means and obturator over wide range of the cannulas of various species. Moreover, the simultaneous presence in dilating trocar 240 both obturator 262 and actuating means 277 provides additional reduction of the operation time. Otherwise, the design and operation of dilating trocar 240 to a large extent are identical to one of dilating trocars 40 and 140, and designations of their identical parts have the same two last numerals.

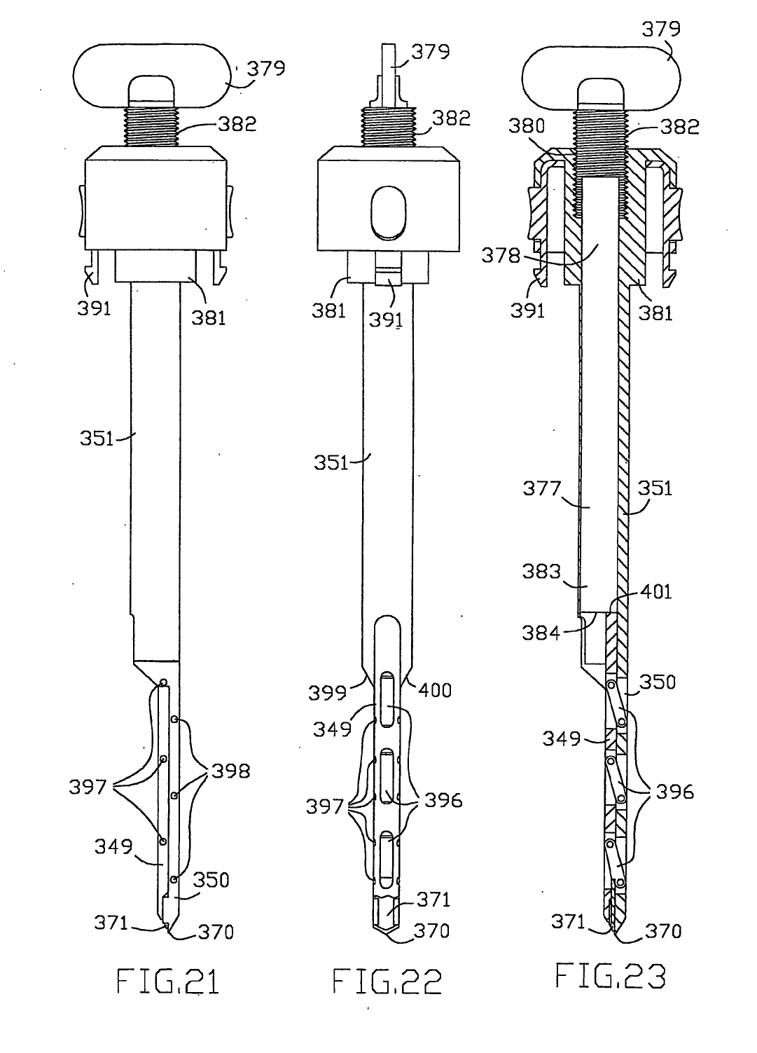

In another version embodiment, shown in FIGS. 21 to 27, dilating trocar 340 comprises carrier 351 removably housed inside cannula 341 and connected with cannula housing 343 by latch 391. Carrier 351 is immovably connected with dilating member 350, which has the penetrating means at its distal end including sharp element 370 and axially movable protective shield 371. Another dilating member 349 is movably connected to immovable dilating member 350 by turning links 396 attached to dilating members 349, 350 through hinges 397, 398 respectively. Proximal face 401 of movable dilating member 349 interacts with distal face 384 of transmitting rod 383 of actuating means 377. The latter is somewhat eccentrically housed inside carrier 351 and rotatably connected with it by thread connection 380. In the first position (FIG. 24), dilating members 349, 350 are tight against each other and sharp element 370 is open allowing the surgeon to form the primary opening and insert the dilating members therein applying insignificant axial effort. Thereafter, the surgeon rotates actuating handle 379 thereby displacing distal face 384 distally and forcing movable dilating member 349 to move in the transversal direction to the second position and stretch the primary opening maintaining the parallelism of dilating members 349, 350 (FIGS. 25, 27). As a result, forcing apart of dilating members 349, 350 does not create any axial forces acting onto the body cavity wall. In doing so, lower turning link 396 displaces protective shield 371 distally and locks it in the distal position, thereby protecting sharp element 370. In version embodiment (not shown), the penetrating means also includes a biasing spring allowing protecting sharp element 370 immediately after its entering the body cavity. As distinct from dilating trocars 40, 140, 240, dilating trocar 340 has does not comprise the traditional obturator, that simplifies the design and reduces the operation time. During subsequent inserting cannula passageway portion 342 into the stretched primary opening, sloping portions 399, 400 of carrier 351 (FIG. 22) facilitate the cannula advance. After the cannula emplacement, the surgeon disengages cannula 341 and carrier 351 by latch 391 and removes carrier 351 along with dilating members 349, 350 and actuating means 377 from cannula 341. Otherwise, the design and operation of dilating trocar 340 to a large extent are identical to one of dilating trocar 40, and designations of their identical parts have the same two last numerals.

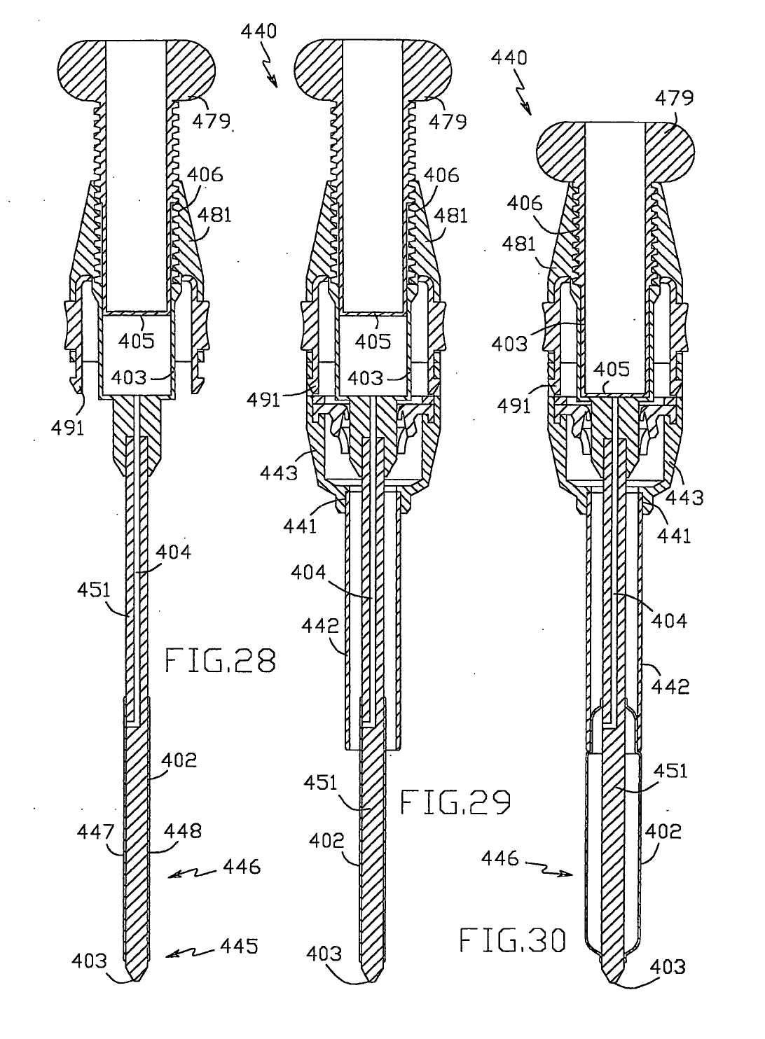

In another version embodiment, shown in FIGS. 28 to 30, dilating trocar 440 comprises cannula 441 with passageway portion 442 and cannula housing 443, carrier 451 with dilating portion 446 made as expandable balloon 402, distal portion 445 and distal blunt penetrating tip 403, and the actuating means including housing 481 connected with cannula housing 443 by controllable latch 491, plunger 405 movably connected with actuating means housing 481 by thread connection 406 an slidably housed in cylinder 405. The cavity of cylinder 405 contains liquid and is communicated with the interior of balloon 402 by channel 404. In the first position (FIG. 29), balloon 402 is empty and tight against carrier 451 forming dilating portion 446 of small cross-sectional area allowing its easy insertion into the primary opening. Thereafter, the surgeon transposes plunger 405 distally forcing out liquid of cylinder 403 into balloon 402 and thereby expanding balloon 402 to the state of the second position (FIG. 30) and stretching the primary opening. Hereafter, the surgeon introduces passageway portion 442 into the stretched primary opening, sucks off liquid from balloon 402 again into cylinder 403 by the axial transposition of plunger 405 proximally, disengages actuating means housing 481 from cannula housing 443 by latch 491, and removes the actuating means along with carrier 451, balloon 402, cylinder 403 and plunger 405 from cannula 441.

1-38. (canceled)

39. A dilating trocar for forming a passageway in a body cavity wall between a body cavity and surroundings by stretching a through primary opening in the body cavity wall to the size of a complete opening, comprising
a cannula having
a housing and;
a tubular passageway portion adapted to the location at the level of said complete opening;
a dilating means designed for stretching said primary opening, and having:
a distal portion, which at least from the beginning to the completion of stretching said primary opening is located inside the body cavity,
a dilating portion of changeable geometry disposed adjacent to said distal portion proximally of it, distally of said passageway portion and having dilating surfaces,
a first position wherein said distal portion is located inside the body cavity, said dilating portion is placed into said primary opening so that at least by the beginning of stretching said primary opening the walls of said primary opening along its full length are disposed within the axial limits of said dilating portion, said dilating surfaces are faced the walls of said primary opening and the transversal dimensions of said dilating portion and distal portion measured at least in the same plane parallel to the longitudinal axis of said dilating trocar are considerably less than the outer diameter of said passageway portion to provide inserting said dilating portion into said primary opening with an insignificant resistance offered by the body tissues and without substantial stretching said primary opening,
a second position wherein said transversal dimensions of said dilating portion are effectively bigger than its correspondent dimensions in said first position, and the transition from said first position to said second position leads to stretching said primary opening to the size approximately equal to the size of said complete opening and sufficient for introducing therein said passageway portion,
an actuating means for transpositions of said dilating means from said first position to said second position.

40. The dilating trocar of claim 39, having
a carrier having
a distal end and;
a proximal end with a housing;
at least one movable dilating member connected to said distal end.

41. The dilating trocar of claim 40, wherein there is a penetrating means for carrying out said primary opening, which in said first position protrudes distally of said distal portion of dilating means.

42. The dilating trocar of claim 41, wherein said penetrating means comprises a sharp element.

43. The dilating trocar of claim 42, wherein said penetrating means has a protecting means for the protection of said sharp element, including a shield and a biasing spring.

44. The dilating trocar of claim 43, wherein said penetrating means is based on said carrier.

45. The dilating trocar of claim 40, wherein there are
a guide for inserting said dilation portion into said primary opening;
said dilating portion comprises an axial passage, wherein said guide is housed during inserting said dilation portion into said primary opening.

46. The dilating trocar of claim 45, wherein said guide is made as a removable obturator.

47. The dilating trocar of claim 40, wherein said carrier is adapted for placement inside said cannula passageway portion and has a carrier attachment means allowing removable attaching said carrier to said cannula.

48. The dilating trocar of claim 47, wherein said carrier attachment means is made as a controllable latch mechanism allowing engagement/disengagement of said carrier and cannula housing.

49. The dilating trocar of claim 40, wherein said cannula fulfils the role of said carrier and said dilating members are connected to a distal end of said cannula passageway portion.

50. The dilating trocar of claim 39, wherein, there is a longitudinal trocar axis and in said first position, said dilating surfaces and said longitudinal trocar axis are substantially parallel to each other.

51. The dilating trocar of claim 50, wherein said dilating surfaces movement occurs mainly in the transversal direction with respect to said longitudinal axis.

52. The dilating trocar of claim 51, wherein said dilating means having
said dilating portion including an expandable balloon and;
said actuating means made as a balloon actuating means designed for expanding said balloon from said first position to said second position by its filling with fluid after said balloon placement into said primary opening.

53. The dilating trocar of claim 52, wherein said balloon actuating means is made as a cylinder containing said fluid in the form of liquid, communicated with said balloon and having a plunger slidably housed inside said cylinder and adapted to forcing out said liquid from said cylinder into said balloon to expand it and to permitting said liquid to revert from said balloon again into said cylinder to collapse said balloon.

54. A dilating trocar for forming a passageway in a body cavity wall between a body cavity and surroundings by stretching a through primary opening in the body cavity wall to the size of a complete opening, comprising
a cannula having a tubular passageway portion adapted to the location at the level of said complete opening and a proximally disposed housing,
a penetrating means for forming said primary opening,
a dilating means designed for stretching said primary opening, and having:
a distal portion, which at least from the beginning to the completion of said primary opening stretching is located inside the body cavity,
a dilating portion of changeable geometry disposed adjacent to said distal portion proximally of it and distally of said passageway portion, and
having dilating surfaces whose length is not less than the thickness of the body cavity wall,
a first position wherein said distal portion is located inside the body cavity, said dilating portion is placed into said primary opening so that at least by the beginning of stretching said primary opening the walls of said primary opening along its full length are disposed within the axial limits of said dilating portion, said penetrating means protrudes distally of said distal portion, said dilating surfaces are faced the walls of said primary opening, and the transversal dimensions of said dilating portion and distal portion measured at least in the same plane parallel to the longitudinal axis of said dilating trocar are considerably less than the outer diameter of said passageway portion to provide inserting said dilating portion into said primary opening with an insignificant resistance offered by a body tissues and without substantial stretching said primary opening, while said cannula passageway portion is located beyond the body cavity wall above it,
a second position, obtained only after said first position, wherein said transversal dimensions of said dilating portion are effectively bigger than its correspondent dimensions in said first position, and the transition from said first position to said second position leads to stretching said primary opening approximately to the size of said complete opening sufficient for introducing therein said passageway portion,
actuating means for transpositions of said dilating means from said first position to said second position.

55. A method for forming a passageway in a body cavity wall between a body cavity and surroundings performed by a dilating trocar set comprising: a cannula with a housing and a tubular passageway portion; a penetrating means, a dilating means comprising an actuating means, a distal portion, and a dilating portion having an extendable transversal dimensions, protruding distally of said cannula passageway portion, therewith in the initial state a cross-sectional area within the limits of an overall outer contour of said dilating and distal portions along their full length is considerably less than one of said cannula passageway portion, said method, comprising:
performing a through relative small primary opening in the body cavity wall by said penetrating means so that, in the following, the dimensions of said primary opening allows inserting therein said dilating portion with insignificant resistance offered by the body tissue and without substantial stretching said primary opening,
inserting said dilating portion into said primary opening by means of said guide so that said distal portion is located within the body cavity, while said cannula passageway tubular portion is located beyond the body cavity wall above it,
fulfilling said complete opening by extending said extendable transversal dimensions of said dilating portion in the transversal direction by said actuating means, thereby stretching said primary opening to a complete opening sufficient for inserting therein said cannula passageway portion,
inserting said cannula passageway portion into said complete opening,
final operations including removing at least said actuating means from an interior of said cannula.

56. The method of claims 55, wherein fulfilling said primary opening and said inserting of said dilating portion into primary opening are accomplished as a single operation.

57. The method of claim 55, performed by said dilating trocar set wherein said dilating portion includes a balloon expandable in the transversal direction and an actuating means in the form of the system for controllable fluid supply into said balloon,
said method, wherein:
said extending said extendable transversal dimension of said dilating portion is accomplished by supplying a pressurized fluid into said balloon leading to expanding said balloon and stretching said primary opening to said complete opening,
said final operations include at least partial removing said fluid from said balloon and removing it from said cannula.
